(19) United States
(12) Patent Application Publication
(10) Pub. No.: US 2008/0125397 A1
(43) Pub. Date: May 29, 2008

(54) 2 (SULFONYL)ETHYL N,N,N',N' tetrakis(2 chloroethyl)phosphorodia

(75) Inventors: Stella K. Lui, Pacifica, CA (US); Wenli Ma, Mountain View, CA (US); Robert M. Yee, San Francisco, CA (US)

Correspondence Address: FOLEY & LARDNER LLP, 975 PAGE MILL ROAD, PALO ALTO, CA, 94304, US

(73) Assignee: Telik, Inc.

(21) Appl. No.: 11/564,091

(22) Filed: Nov. 28, 2006

(51) Int. Cl. A61K 31/675; A61P 35/04; C07F 9/06

(52) U.S. Cl. 514/89; 546/22; 564/12

(57) ABSTRACT

2-({2-Oxo-2-[(pyridin-3-ylmethyl)amino]ethyl}sulfonyl)ethyl N,N,N′,N′-tetrakis(2-chloroethyl)phosphorodiamidate and its acid addition salts, especially in solid form, their preparation and intermediates in their preparation, formulations containing them, and methods of treatment using them. The compounds are useful for treating cancer, alone and in combination with other anticancer therapies.

### BACKGROUND OF THE INVENTION

1. Field of the Invention

This invention relates to 2-({2-oxo-2-[(pyridin-3-ylmethyl)amino]ethyl}sulfonyl)ethyl N,N,N′,N′-tetrakis(2-chloroethyl)phosphorodiamidate and its acid addition salts, their preparation and intermediates in their preparation, formulations containing them, and methods of treatment using them.

2. Description of the Related Art

U.S. Pat. No. 5,556,942 [and PCT Publication No. WO 95/09865] discloses compounds of the formula

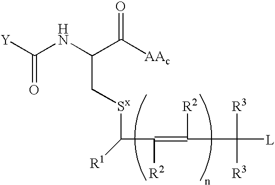

and their amides, esters, and salts, where:
L is an electron withdrawing leaving group;
S^{x} is —S(═O)—, —S(═O)₂—, —S(═NH)—, —S(═O)(═NH)—, —S⁺(C₁-C₆ alkyl)-, —Se(═O)—, —Se(═O)₂—, —Se(═NH)—, or —Se(═O)(═NH)—, or is —O—C(═O)—, or —HN—C(═O)—;
each R¹, R² and R³ is independently H or a non-interfering substituent;
n is 0, 1 or 2;
Y is selected from the group consisting of

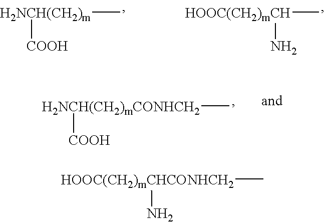

where m is 1 or 2; and
AA_{c} is an amino acid linked through a peptide bond to the remainder of the compound.

The compounds are stated to be useful drugs for the selective treatment of target tissues which contain compatible GST isoenzymes, and simultaneously elevate the levels of GM progenitor cells in bone marrow. Disclosed embodiments for L include those that generate a drug that is cytotoxic to unwanted cells, including the phosphoramidate and phosphorodiamidate mustards.

One of the compounds has the formula

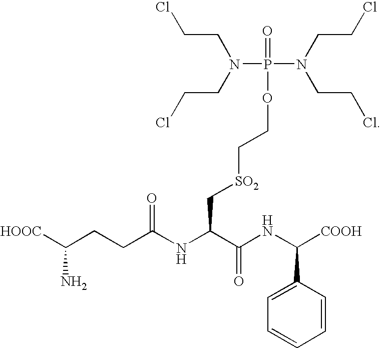

It is referred to in the patent as TER 286 and named as γ-glutamyl-α-amino-β-((2-ethyl-N,N,N,N-tetra(2′-chloro)ethylphosphoramidate)sulfonyl)propionyl-(R)-(-)-phenylglycine. This compound, now referred to as TLK286, has the uninverted CAS name L-γ-glutamyl-3-[[2-[[bis[bis(2-chloroethyl)amino]phosphinyl]oxy]ethyl]sulfonyl]-L-alanyl-2-phenyl-(2R)-glycine. As the neutral compound, its recommended International Nonproprietary Name is canfosfamide; and as its hydrochloride acid addition salt, its United States Adopted Name is canfosfamide hydrochloride. Canfosfamide and its salts are anticancer compounds that are activated by the actions of GST P1-1, and by GST A1-1, to release the cytotoxic phosphorodiamidate mustard moiety.

In vitro, canfosfamide has been shown to be more potent in the M6709 human colon carcinoma cell line selected for resistance to doxorubicin and the MCF-7 human breast carcinoma cell line selected for resistance to cyclophosphamide, both of which overexpress GST P1-1, over their parental cell lines; and in murine xenografts of M7609 engineered to have high, medium, and low levels of GST P1-1, the potency of canfosfamide hydrochloride was positively correlated with the level of GST P1-1 (Morgan et al., “Tumor efficacy and bone marrow-sparing properties of TER286, a cytotoxin activated by glutathione S-transferase”, Cancer Res., 58, 2568-2575 (1998)).

Canfosfamide hydrochloride is currently being evaluated in multiple clinical trials for the treatment of ovarian, breast, non-small cell lung, and colorectal cancers. It has demonstrated significant single agent antitumor activity and improvement in survival in patients with non-small cell lung cancer and ovarian cancer, and single agent antitumor activity in colorectal cancer and breast cancer. Evidence from in vitro cell culture and tumor biopsies indicates that canfosfamide is non-cross-resistant to platinum, paclitaxel, and doxorubicin (Rosario et al., “Cellular response to a glutathione S-transferase P1-1activated prodrug”, Mol. Pharmacol., 58, 167-174 (2000)), and also to gemcitabine. Patients treated with canfosfamide hydrochloride show a very low incidence of clinically significant hematological toxicity.

PCT Publication No. WO 95/09865 also discloses intermediates that are compounds of the formula

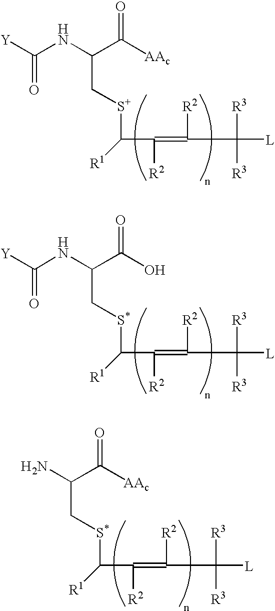

and their amides, esters, and salts, where:
L is an electron withdrawing leaving group;
S⁺ is S or Se;
S* is —S(═O)—, —S(═O)₂—, —S(═NH)—, —S(═O)(═NH)—, —S⁺(C₁-C₆ alkyl)-, —Se(═O)—, —Se(═O)₂—, —Se(═NH)—, or —Se(═O)(═NH)—, or is —O—C(═O)—, or —HN—C(═O)—;
each R¹, R² and R³ is independently H or a non-interfering substituent;
n is 0, 1 or2;

### Y is selected from the group consisting of

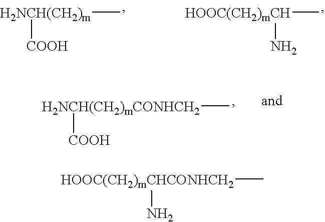

where m is 1 or 2; and
AA_{c} is an amino acid linked through a peptide bond to the remainder of the compound.

U.S. Pat. No. 6,506,739 [and PCT Publication No. WO 01/83496] discloses compounds of the formula

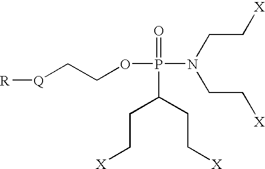

where:
X is a halogen atom;
Q is O, S, or NH; and
R is hydrogen, optionally substituted lower alkyl, optionally substituted aryl, or optionally substituted heteroaryl, or is R′CO—, R′NHCO—, R′SO₂—, or R′NHSO₂— where R′ is hydrogen, optionally substituted lower alkyl, optionally substituted aryl, or optionally substituted heteroaryl; or
R-Q together is chlorine;
and their salts,
as antitumor agents.

US Patent Application Publication No. 2005/0267075 [and PCT Publication No. WO 2005/118601] discloses compounds of the formulae

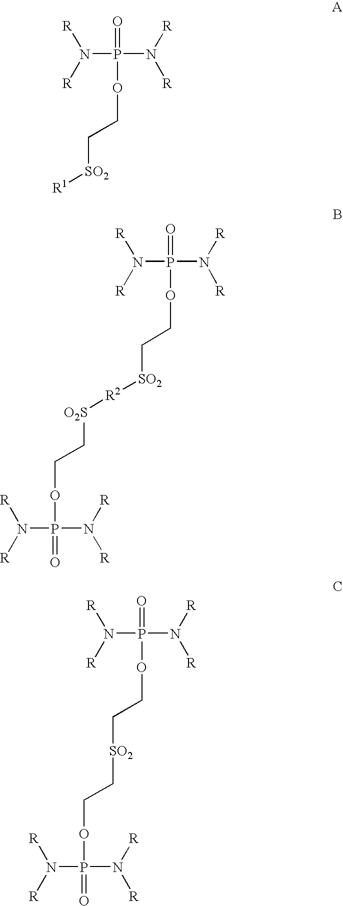

where:
each R is independently hydrogen, C₁₋₆ alkyl, or —CH₂CH₂X, where each X is independently Cl, Br, C₁₋₆ alkanesulfonyloxy, halo-C₁₋₆ alkanesulfonyloxy, or benzenesulfonyloxy optionally substituted with up to three substituents selected from halo, C₁₋₃ alkyl, halo-C₁₋₃ alkyl, C₁₋₃ alkyloxy, or halo-C₁₋₃ alkyloxy, provided that at least two R's in each phosphorodiamidate group are —CH₂CH₂X;
R¹ is optionally substituted alkyl, optionally substituted heteroalkyl, optionally substituted aryl, optionally substituted aralkyl, optionally substituted heteroaryl, or optionally substituted heteroaralkyl; and
R²is optionally substituted alkanediyl, optionally substituted heteroalkanediyl, optionally substituted arenediyl, optionally substituted arenedialkyl, optionally substituted heteroarenediyl, or optionally substituted heteroarenedialkyl,
and their salts,
as antitumor agents.

Jain et al., “Sulfonyl-containing aldophosphamide analogues as novel anticancer prodrugs targeted against cyclophosphamide-resistant tumor cell lines”, J. Med. Chem., 47(15), 3843-3852 (2004), discloses a series of sulfonylethyl phosphorodiamidates of the formula

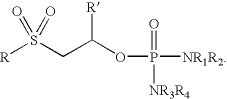

The compounds are said to spontaneously liberate phosphoramide mustards via beta-elimination, and to be more potent than the corresponding phosphoramide mustards against V-79 Chinese hamster lung fibroblasts in vitro. Some of the compounds were said to show excellent in vivo antitumor activity in CD2F1 mice against the P388/0 (wild) and P388/CPA (cyclophosphamide-resistant) leukemia cell lines.

It would be desirable to develop a chemically and pharmaceutically simple (easy to synthesize and formulate) anticancer drug having an efficacy and safety as good or better than canfosfamide.

The disclosures of the documents referred to in this application are incorporated into this application by reference.

### SUMMARY OF THE INVENTION

In a first aspect, this invention is 2-({2-oxo-2-[(pyridin-3-ylmethyl)amino]ethyl}sulfonyl)-ethyl N,N,N′,N′-tetrakis(2-chloroethyl)phosphorodiamidate and its acid addition salts, preferably in solid form.

In a second aspect, this invention is pharmaceutical compositions comprising a compound of the first aspect of this invention.

In a third aspect, this invention is methods of treating cancer by the administration of a compound of the first aspect of this invention or a pharmaceutical composition of the second aspect of this invention; alone or in combination with other anticancer therapies.

In a fourth aspect, this invention is methods of preparing compounds of the first aspect of this invention and intermediates in their preparation.

### DETAILED DESCRIPTION OF THE INVENTION

Definitions

“Acid addition salts” are described in the section entitled “Compounds of this invention”.

A “therapeutically effective amount” means that amount which, when administered to a human for treating a cancer, is sufficient to effect treatment for the cancer. “Treating” or “treatment” of a cancer in a human includes one or more of:
(1) limiting/inhibiting growth of the cancer, i.e., limiting/arresting its development,
(2) reducing/preventing spread of the cancer, i.e. reducing/preventing metastases,
(3) relieving the cancer, i.e., causing regression of the cancer,
(4) reducing/preventing recurrence of the cancer, and
(5) palliating symptoms of the cancer.

“Combination therapy” means the administration of a compound of the first aspect of this invention (e.g. in the form of a composition containing it) and another anticancer therapy during the course of cancer chemotherapy. Such combination therapy may involve the administration of the compound of the first aspect of this invention before, during, and/or after the administration of the another anticancer therapy. The administration of the compound of the first aspect of this invention may be separated in time from the administration of the another anticancer therapy by up to several weeks, and may precede it or follow it, but more commonly the administration of the compound of the first aspect of this invention will accompany at least one aspect of the another anticancer therapy (such as the administration of one dose of a chemotherapeutic agent, molecular targeted therapy agent, biologic therapy agent, or radiation therapy) within up to 48 hours, and most commonly within less than 24 hours.

“Another anticancer therapy” is an anticancer therapy that is not a treatment with a compound of the first aspect of this invention. Such “another anticancer therapies” include chemotherapy; molecular targeted therapy; biologic therapy; and radiotherapy. These therapies are those used as monotherapy or in combination therapy.

Chemotherapeutic Agents Include:
alkylating agents, including:
alkyl sulfonates such as busulfan,
ethyleneimine derivatives such as thiotepa,
nitrogen mustards such as chlorambucil, cyclophosphamide, estramustine, ifosfamide, mechlorethamine, melphalan, and uramustine,
nitrosoureas such as carmustine, lomustine, and streptozocin,
triazenes such as dacarbazine, procarbazine, and temozolamide, and platinum compounds such as cisplatin, carboplatin, oxaliplatin, satraplatin, and picoplatin;
antimetabolites, including:
antifolates such as methotrexate, permetrexed, raltitrexed, and trimetrexate,
purine analogs such as cladribine, chlorodeoxyadenosine, clofarabine, fludarabine, mercaptopurine, pentostatin, and thioguanine,
pyrimidine analogs such as azacitidine, capecitabine, cytarabine, edatrexate, floxuridine, fluorouracil, gemcitabine, and troxacitabine;
natural products, including:
antitumor antibiotics such as bleomycin, dactinomycin, mithramycin, mitomycin, mitoxantrone,
porfiromycin, and anthracyclines such as daunorubicin (including liposomal daunorubicin),
doxorubicin (including liposomal doxorubicin), epirubicin, idarubicin, and valrubicin,
enzymes such as L-asparaginase and PEG-L-asparaginase,
microtubule polymer stabilizers such as the taxanes paclitaxel and docetaxel,
mitotic inhibitors such as the vinca alkaloids vinblastine, vincristine, vindesine, and vinorelbine,
topisomerase I inhibitors such as the camptothecins irinotecan and topotecan, and topoisomerase II inhibitors such as amsacrine, etoposide, and teniposide;
hormones and hormone antagonists, including:
androgens such as fluoxymesterone and testolactone,
antiandrogens such as bicalutamide, cyproterone, flutamide, and nilutamide,
aromatase inhibitors such as aminoglutethimide, anastrozole, exemestane, formestane, and letrozole,
corticosteroids such as dexamethasone and prednisone,
estrogens such as diethylstilbestrol,
antiestrogens such as fulvestrant, raloxifene, tamoxifen, and toremifine,
LHRH agonists and antagonists such as buserelin, goserelin, leuprolide, and triptorelin,
progestins such as medroxyprogesterone acetate and megestrol acetate, and thyroid hormones such as levothyroxine and liothyronine; and
miscellaneous agents, including altretamine, arsenic trioxide, gallium nitrate, hydroxyurea, levamisole, mitotane, octreotide, procarbazine, suramin, thalidomide, lenalidomide, photodynamic compounds such as methoxsalen and sodium porfimer, and proteasome inhibitors such as bortezomib.

Molecular Targeted Therapy Agents Include:
functional therapeutic agents, including:
gene therapy agents,
antisense therapy agents,
tyrosine kinase inhibitors such as erlotinib hydrochloride, gefitinib, imatinib mesylate, and semaxanib, and
gene expression modulators such as the retinoids and rexinoids, e.g. adapalene, bexarotene, trans-retinoic acid, 9-cis-retinoic acid, and N-(4-hydroxyphenyl)retinamide;
phenotype-directed therapy agents, including:
monoclonal antibodies such as alemtuzumab, bevacizumab, cetuximab, ibritumomab tiuxetan, rituximab, and trastuzumab,
immunotoxins such as gemtuzumab ozogamicin, and
radioimmunoconjugates such as ¹³¹I-tositumomab; and
cancer vaccines.

Biologic Therapy Agents Include:
interferons such as interferon-α₂ₐ and interferon-α_{2b}, and
interleukins such as aldesleukin, denileukin diftitox, and oprelvekin.

In addition to these agents intended to act against cancer cells, anticancer therapies include the use of protective or adjunctive agents, including:
cytoprotective agents such as amifostine, dexrazoxane, and mesna,
phosphonates such as pamidronate and zoledronic acid, and
stimulating factors such as epoetin, darbeopetin, filgrastim, PEG-filgrastim, and sargramostim.

Combination cancer therapy regimens with which the compounds of the first aspect of this invention may be combined include all regimens involving the use of two or more of the anticancer therapies (anticancer agents) such as those mentioned in paragraphs [0022] to [0024] above and/or radiotherapy, optionally including protective and adjunctive agents such as those mentioned in paragraph [0025] above; and the compound of the first aspect of this invention can be added to existing anticancer regimens known for the treatment of various cancers, such as the regimens mentioned in such books as Chabner and Longo, eds., “Cancer Chemotherapy and Biotherapy: Principles and Practice”, 3rd ed. (2001), and Skeel, ed., “Handbook of Cancer Chemotherapy”, 6^{th} ed. (2003), both from Lippincott Williams & Wilkins, Philadelphia, Pa., U.S.A.; and regimens for anticancer therapies, especially chemotherapies, may be found on Web sites such as those maintained by the National Cancer Institute (www.cancer.gov), the American Society for Clinical Oncology (www.asco.org), and the National Comprehensive Cancer Network (www.nccn.org).

Many combination chemotherapeutic regimens are known to the art, such as combinations of platinum compounds and taxanes, e.g. carboplatin/paclitaxel, capecitabine/docetaxel, the “Cooper regimen”, fluorouracil-levamisole, fluorouracil-leucovorin, methotrexate-leucovorin, and those known by the acronyms ABDIC, ABVD, AC, ADIC, AI, BACOD, BACOP, BVCPP, CABO, CAD, CAE, CAF, CAP, CD, CEC, CF, CHOP, CHOP+rituximab, CIC, CMF, CMFP, CyADIC, CyVADIC, DAC, DVD, FAC, FAC-S, FAM-S, FOLFOX-4, FOLFOX-6, M-BACOD, MACOB-B, MAID, MOPP, MVAC, PCV, T-5, VAC, VAD, VAPA, VAP-Cyclo, VAP-II, VBM, VBMCP, VIP, VP, and the like.

Combinations of chemotherapies and molecular targeted therapies, biologic therapies, and radiation therapies are also well known to the art; including therapies such as trastuzumab+paclitaxel, alone or in further combination with carboplatin, for certain breast cancers, and many other such regimens for other cancers; and the “Dublin regimen” and “Michigan regimen”, both for esophageal cancer, and many other such regimens for other cancers.

“Comprising” or “containing” and their grammatical variants are words of inclusion and not of limitation and mean to specify the presence of stated components, groups, steps, and the like but not to exclude the presence or addition of other components, groups, steps, and the like. Thus “comprising” does not mean “consisting of”, “consisting substantially of”, or “consisting only of”; and, for example, a formulation “comprising” a compound must contain that compound but may also may contain other active ingredients and/or excipients.

Compounds of This Invention

In a first aspect, this invention is 2-({2-oxo-2-[(pyridin-3-ylmethyl)amino]ethyl}sulfonyl)-ethyl N,N,N′,N′-tetrakis(2-chloroethyl)phosphorodiamidate and its acid addition salts.

Acid addition salts (for example, pharmaceutically acceptable acid addition salts) of 2-{2-oxo-2-[(pyridin-3-ylmethyl)amino]ethylsulfonyl}ethyl N,N,N′,N′-tetrakis(2-chloroethyl)-phosphorodiamidate are included in the present invention and are useful in the compositions, methods, and uses described in this application. Suitable salts are those formed when inorganic acids (e.g. hydrochloric, hydrobromic, sulfuric, nitric, phosphoric, and chlorosulfonic acids) or organic acids (e.g. acetic, propionic, oxalic, malic, maleic, malonic, fumaric, citric, tartaric, lactic, succinic, and aceturic acids, and alkane- or arenesulfonic acids such as methanesulfonic, ethanesulfonic, 2-hydroxyethanesulfonic, benzenesulfonic, substituted benzenesulfonic such as chlorobenzenesulfonic and toluenesulfonic, naphthalenesulfonic and substituted naphthalene-sulfonic, naphthalenedisulfonic and substituted naphthalenedisulfonic, and camphorsulfonic acids) react to form acid addition salts of the amine groups of the compounds. Such salts are preferably formed with pharmaceutically acceptable acids. See, for example, Stahl and Wermuth, eds., “Handbook of Pharmaceutically Acceptable Salts”, (2002), Verlag Helvetica Chimica Acta, Zürich, Switzerland, for an extensive discussion of pharmaceutical salts, their selection, preparation, and use.

Preparation of The Compounds

2-({2-Oxo-2-[(pyridin-3-ylmethyl)amino]ethyl}sulfonyl)ethyl N,N,N′,N′-tetrakis(2-chloroethyl)phosphorodiamidate may conveniently be prepared by:
(1) preparing 2-[(carboxymethyl)sulfonyl]ethyl N,N,N′,N′-tetrakis(2-chloroethyl)-phosphorodiamidate, followed by reacting this intermediate with 3-(aminomethyl)pyridine [Method 1—as illustrated in the Synthesis Example], or
(2) preparing 2-({2-oxo-2-[(pyridin-3-ylmethyl)amino]ethyl}thio)ethyl N,N,N′,N′-tetrakis(2-chloroethyl)phosphorodiamidate, followed by oxidizing the sulfide [Method 2], as illustrated below.

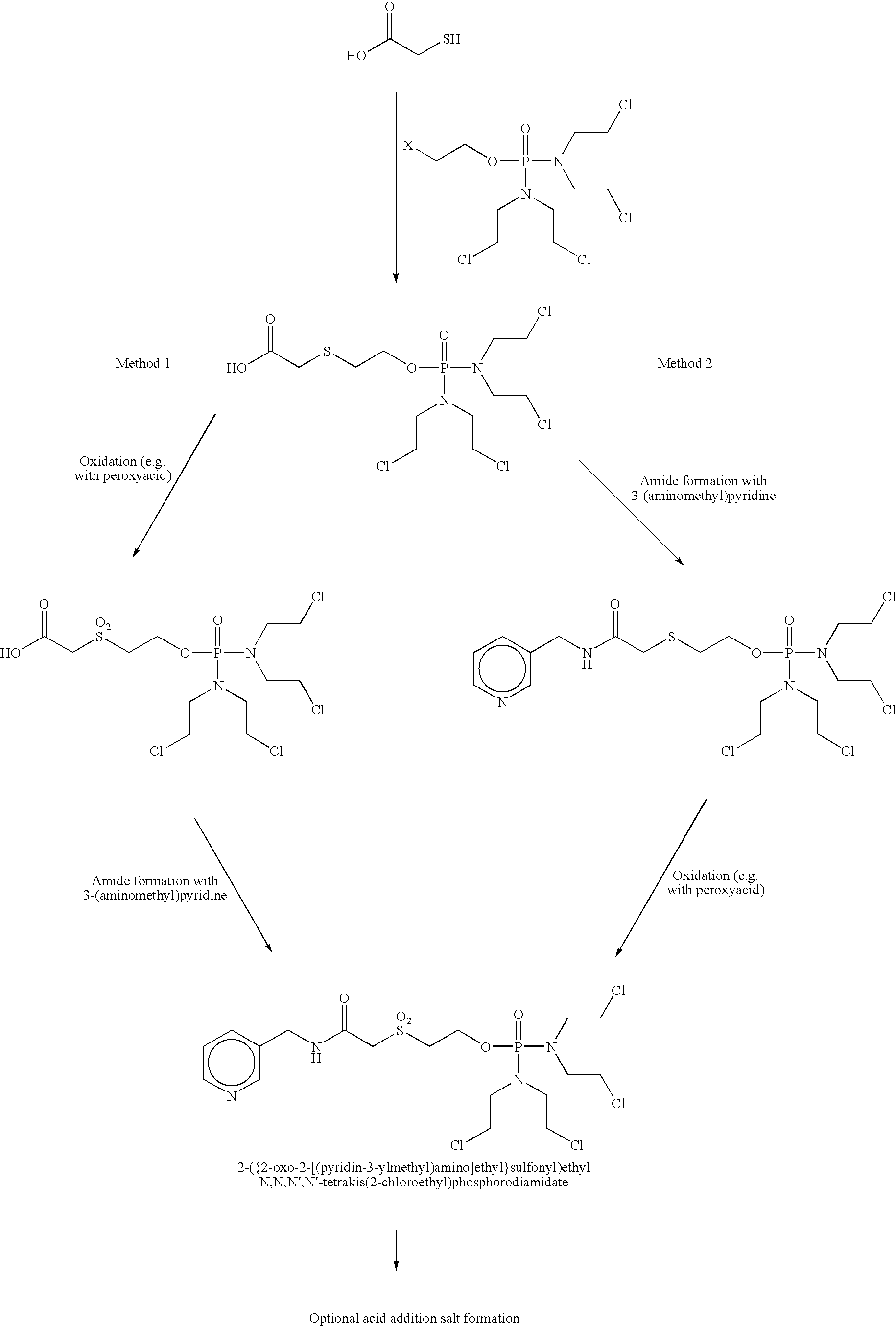

In the first step of either method, thioglycolic acid is converted into 2-[(carboxymethyl)-thio]ethyl N,N,N′,N′-tetrakis(2-chloroethyl)phosphorodiamidate by reaction with a 2-X-ethyl N,N,N′,N′-tetrakis(2-chloroethyl)phosphorodiamidate (where X is a leaving group such as Cl, Br, C₁₋₆ alkanesulfonyloxy, halo-C₁₋₆ alkanesulfonyloxy, or benzenesulfonyloxy optionally substituted with up to three substituents selected from halo, C₁₋₃ alkyl, halo-C₁₋₃ alkyl, C₁₋₃ alkyloxy, or halo-C₁₋₃ alkyloxy, such as methanesulfonyloxy, benzenesulfonyloxy, 4-bromobenzenesulfoxy, or 4-toluenesulfonyloxy). A typical procedure involves treating the thioglycolic acid with a polar solvent such as water, an alkanol, dimethylformamide, or tetrahydrofuran, and a base such as a hydroxide, alkoxide, fluoride, hydride, or a tertiary amine or amide base to form the thiolate anion of the acid, followed by adding the phosphorodiamidate. Thiolate displacement of the leaving group X of the phosphorodiamidate gives 2-[(carboxymethyl)thio]ethyl N,N,N′,N′-tetrakis(2-chloroethyl)-phosphorodiamidate.

In the second step of Method 1, the 2-[(carboxymethyl)thio]ethyl N,N,N′,N′-tetrakis(2-chloroethyl)phosphorodiamidate is oxidized to 2-[(carboxymethyl)sulfonyl]-ethyl N,N,N′,N′-tetrakis(2-chloroethyl)phosphorodiamidate. This oxidation may be performed by any of the methods known in the art for the oxidation of sulfides to sulfones, such as the use of peracids (peroxycarboxylic acids), persulfates, perborates, peroxides, ozone, iodosyl reagents, halogens, and the like. Where a peracid is used, a typical procedure involves dissolving the 2-[(carboxymethyl)-thio]ethyl N,N,N′,N′-tetrakis(2-chloroethyl)phosphorodiamidate in a solvent such as dichloromethane, acetic acid, or isopropyl acetate at reduced temperature, followed by the addition of the peracid (e.g. peracetic acid) in excess.

In the third step of Method 1, the 2-[(carboxymethyl)sulfonyl]ethyl N,N,N′,N′-tetrakis(2-chloroethyl)phosphorodiamidate is reacted with 3-(aminomethyl)pyridine to form 2-({2-oxo-2-[(pyridin-3-ylmethyl)amino]ethyl}sulfonyl)ethyl N,N,N′,N′-tetrakis(2-chloroethyl)phosphorodiamidate. This reaction may be performed by any of the methods known in the art for the formation of amides from carboxylic acids and secondary amines, typically by activating the carboxylic acid (in solution in a strongly polar solvent such as dimethylformamide) with an activating agent such as a uronium, phosphonium, or pyridinium salt, carbodiimide, or active ester, followed by reaction with an excess of the 3-(aminomethyl)pyridine in the presence of an excess of a tertiary amine base.

In the second step of Method 2, the 2-[(carboxymethyl)thio]ethyl N,N,N′,N′-tetrakis(2-chloroethyl)phosphorodiamidate is reacted with 3-(aminomethyl)pyridine to form 2-({2-oxo-2-[(pyridin-3-ylmethyl)amino]ethyl}thio)ethyl N,N,N′,N′-tetrakis(2-chloroethyl)-phosphorodiamidate. This reaction may be performed by any of the methods described for the third step of Method 1 in paragraph [0037].

In the third step of Method 2, the 2-({2-oxo-2-[(pyridin-3-ylmethyl)amino]ethyl}thio)ethyl N,N,N′,N′-tetrakis(2-chloroethyl)phosphorodiamidate is oxidized to 2-({2-oxo-2-[(pyridin-3-yl-methyl)amino]ethyl}sulfonyl)ethyl N,N,N′,N′-tetrakis(2-chloroethyl)phosphorodiamidate. This oxidation may be performed by any of the methods described for the second step of Method 1 in paragraph [0036]; however the oxidation should be performed under conditions that minimize oxidation of the pyridine nitrogen, such as by performing the oxidation at a sufficiently low pH to stabilize the pyridine as a pyridinium cation.

The 2-({2-oxo-2-[(pyridin-3-ylmethyl)amino]ethyl}sulfonyl)ethyl N,N,N′,N′-tetrakis(2-chloroethyl)phosphorodiamidate may be converted to an acid addition salt by reaction with the appropriate acid, using techniques well known to a person of ordinary skill in the art for the formation of acid addition salts. The acid used, and the reaction conditions, may be chosen to give an acid addition salt that is pharmaceutically acceptable and that has a form convenient for isolation and formulation, such as a solid form (for example, amorphous or crystalline).

Pharmaceutical Compositions and Administration

The second aspect of this invention is pharmaceutical compositions comprising a compound of the first aspect of this invention and optionally a pharmaceutically acceptable excipient.

The compounds of the first aspect of this invention may be administered by any route suitable to the subject being treated and the nature of the subject's condition. Routes of administration include administration by injection, including intravenous, intraperitoneal, intramuscular, and subcutaneous injection, by transmucosal or transdermal delivery, through topical applications, nasal spray, suppository and the like or may be administered orally. Formulations may optionally be liposomal formulations, emulsions, formulations designed to administer the drug across mucosal membranes or transdermal formulations. Suitable formulations for each of these methods of administration may be found, for example, in Gennaro, ed., “Remington: The Science and Practice of Pharmacy”, 20th ed. (2000), Lippincott Williams & Wilkins, Philadelphia, Pa., U.S.A. Typical formulations will be either oral or solutions for intravenous infusion. Typical dosage forms will be tablets (including coated tablets and “caplets”) or capsules (including hard gelatin capsules and “softgels”) for oral administration, solutions for intravenous infusion, and solids (especially lyophilized powders) for reconstitution as solutions for intravenous infusion.

Depending on the intended mode of administration, the pharmaceutical compositions may be in the form of solid, semi-solid or liquid dosage forms, preferably in unit dosage form suitable for single administration of a precise dosage. In addition to an effective amount of the active ingredient(s), the compositions may contain suitable pharmaceutically-acceptable excipients, including adjuvants which facilitate processing of the active compounds into preparations which can be used pharmaceutically. “Pharmaceutically acceptable excipient” refers to an excipient or mixture of excipients which does not interfere with the effectiveness of the biological activity of the active ingredient(s) and which is not toxic to the host to which it is administered.

For solid compositions, conventional excipients include, for example, pharmaceutical grades of mannitol, lactose, starch, magnesium stearate, sodium saccharin, talc, cellulose, glucose, sucrose, magnesium carbonate, and the like. Liquid pharmacologically administrable compositions can, for example, be prepared by dissolving, dispersing, etc., an active compound and optional pharmaceutical adjuvants in an excipient, such as, for example, water, saline, aqueous dextrose, glycerol, ethanol, and the like, to thereby form a solution or suspension. If desired, the pharmaceutical composition to be administered may also contain minor amounts of nontoxic auxiliary excipients such as wetting or emulsifying agents, pH buffering agents and the like, for example, sodium acetate, sorbitan monolaurate, triethanolamine sodium acetate, triethanolamine oleate, etc.

For oral administration, the composition will generally take the form of a tablet or capsule, or it may be an aqueous or nonaqueous solution, suspension or syrup. Tablets and capsules are preferred oral administration forms. Tablets and capsules for oral use will generally include one or more commonly used excipients such as lactose and corn starch. Lubricating agents, such as magnesium stearate, are also typically added. When liquid suspensions are used, the active agent may be combined with emulsifying and suspending excipients. If desired, flavoring, coloring and/or sweetening agents may be added as well. Other optional excipients for incorporation into an oral formulation include preservatives, suspending agents, thickening agents, and the like.

Injectable formulations can be prepared in conventional forms, either as liquid solutions or suspensions, solid forms suitable for dissolution or suspension in liquid prior to injection, or as emulsions or liposomal formulations. The sterile injectable formulation may also be a sterile injectable solution or a suspension in a parenterally pharmaceutically acceptable diluent or solvent. Among the acceptable vehicles and solvents that may be employed are water, Ringer's solution and isotonic sodium chloride solution. In addition, sterile, fixed oils, fatty esters, and polyols are conventionally employed as solvents or suspending media.

The pharmaceutical compositions of this invention may also be formulated as lyophilized powders for parenteral administration. Powders may be reconstituted by addition of water or other primarily aqueous medium and then further diluted with a suitable diluent prior to use. The liquid formulation is generally a buffered, isotonic, aqueous solution. Examples of suitable diluents are isotonic saline solution, aqueous 5% dextrose solution, and buffered sodium or ammonium acetate solution. Pharmaceutically acceptable solid or liquid excipients may be added to enhance or stabilize the composition, or to facilitate preparation of the composition.

Typically, a pharmaceutical composition of the present invention is packaged in a container with a label, or instructions, or both, indicating use of the pharmaceutical composition in the treatment of cancer.

The pharmaceutical composition may additionally contain one or more other pharmacologically active agents in addition to a compound of this invention. These additional active agents will typically be useful in treating cancer, or for enhancing the treatment of cancer by compounds of this invention.

Methods of Using the Compounds

The compounds of the first aspect of this invention have activity against human cancer cell lines, as demonstrated in the in vitro and in vivo Examples below, and are therefore considered to be useful as human cancer chemotherapeutic agents, for the treatment of human cancers.

Thus, the third aspect of this invention includes methods of treating cancer in humans by administering a therapeutically effective amount of a compound of the first aspect of this invention, or a pharmaceutical composition of the second aspect of this invention, to the human. Optionally, the methods further comprise treating the human with another anticancer therapy, such as a therapy already conventional for the cancer being treated.

Cancers that are particularly treatable by the method of this invention are cancers with sensitivity to inducers of apoptosis, and more specifically those cancers that express or, particularly, overexpress one or more glutathione S-transferase isoenzymes. Cancers that express or overexpress one or more glutathione S-transferase isoenzymes when treated with other anticancer compounds or combination cancer chemotherapy regimens are especially treatable by the method of this invention. Such cancers include cancers of the brain, breast, bladder, cervix, colon and rectum, esophagus, head and neck, kidney, lung, liver, ovary, pancreas, prostate, and stomach; leukemias such as ALL, AML, AMML, CLL, CML, CMML, and hairy cell leukemia; Hodgkin's and non-Hodgkin's lymphomas; mesotheliomas, multiple myeloma; and sarcomas of bone and soft tissue. Cancers particularly treatable by the method of this invention include breast, ovarian, colorectal, and non-small cell lung cancers.

The amount of the compound of the first aspect of this invention that is administered to the human (either alone or, more usually, in a composition of the second aspect of this invention) should be a therapeutically effective amount when used alone or when used in conjunction with the another anticancer therapy (if the compound of the first aspect of this invention is administered in conjunction with another anticancer therapy); and similarly the amount of the another anticancer therapy that is administered to the mammal (if the compound of the first aspect of this invention is administered in conjunction with another anticancer therapy) should be a therapeutically effective amount when used in conjunction with the compound of the first aspect of this invention. However, the therapeutically effective amount of either the compound of the first aspect of this invention and the amount of the another anticancer therapy when administered in combination cancer chemotherapy may each be less than the amount which would be therapeutically effective if delivered to the human alone. It is common in cancer therapy, though, to use the maximum tolerated dose of the or each therapy, with a reduction only because of common toxicity of the therapies used or potentiation of the toxicity of one therapy by another. Because of the lack of cross-resistance of canfosfamide, for example, with several common chemotherapeutic agents, and its relative lack of clinically severe toxicity, especially its lack of clinically severe hematological toxicity, it is expected that compounds of the first aspect of this invention will be administrable at essentially their maximum tolerated dose as a single agent, and no reduction in the amount of the another anticancer therapy will be required.

The compounds of the first aspect of this invention, or pharmaceutical compositions of the second aspect of this invention, are thus used to treat cancer in humans requiring such treatment, by administering a therapeutically effective amount of the chosen compound or composition. Therapeutically effective amounts of compounds of the invention are in the range of 10-10,000 mg/m², for example, 30-3000 mg/m² or 100-1000 mg/m². Dosing may be at 1-35 day intervals; for example, about 500-1000 mg/m² at 1-5 week intervals, especially at 1, 2, 3, or 4 week intervals, or at higher frequencies including as frequently as once/day for several (e.g. 5 or 7) days, with the dosing repeated every 2, 3, or 4 weeks, or constant infusion for a period of 6-72 hours, also with the dosing repeated every 2, 3, or 4 weeks. Suitable dosages and dose frequencies will be readily determinable by a person of ordinary skill in the art having regard to that skill and this disclosure. No unacceptable toxicological effects are expected when compounds of the invention are administered in accordance with the present invention.

Suitable dosing for the other anticancer therapy (if the compound of the first aspect of this invention is used in combination) will be the dosing already established for that therapy, as described in such documents as those listed in paragraph [0026]. Such dosing varies widely with the therapy: for example, capecitabine (2500 mg/m² orally) is dosed twice daily for 2 weeks on and 1 week off, imatinib mesylate (400 or 600 mg orally) is dosed daily, rituximab is dosed weekly, paclitaxel (135-175 mg/m²) and docetaxel (60-100 mg/m²) are dosed weekly to every three weeks, carboplatin (4-6 mg/mL·min) is dosed once every 3 or 4 weeks (though the doses may be split and administered over several days), nitrosourea alkylating agents such as carmustine are dosed as infrequently as once every 6 weeks. Radiotherapy may be administered as frequently as weekly (or even within that split into smaller dosages administered daily).

A person of ordinary skill in the art of cancer therapy will be able to ascertain a therapeutically effective amount of the compound of the first or second aspect of this invention and a therapeutically effective amount of another anticancer therapy for a given cancer and stage of disease without undue experimentation and in reliance upon personal knowledge and the disclosure of this application.

Combination therapies include the combination administration of a compound of the first aspect of this invention with a platinum compound such as carboplatin or cisplatin, optionally in further combination with gemcitabine or a taxane such as docetaxel or paclitaxel; with gemcitabine; with a taxane; with an anthracycline such as doxorubicin or liposomal doxorubicin; with oxaliplatin, optionally in further combination with capecitabine or fluorouracil/leucovorin; and with gemcitabine or a platinum compound such as carboplatin or cisplatin, in further combination with a vinca alkaloid such as vinorelbine.

### EXAMPLES

The following examples illustrate the preparation of compounds of this invention, and their activity in predictive in vitro and in vivo anticancer assays.

### Synthesis Example

### Preparation of 2-({2-oxo-2-[(pyridin-3-ylmethyl)amino]ethyl}-sulfonyl)ethyl N,N,N′,N′-tetrakis(2-chloroethyl)phosphorodiamidate as its hydrochloride and tartrate acid addition salts.

The compounds of this invention are prepared by conventional methods of organic chemistry. See, for example, Larock, “Comprehensive Organic Transformations”, (1989), Wiley-VCH, New York, N.Y., U.S.A. The compounds of this invention can be synthesized, generally following the synthetic schemes illustrated earlier in this application, as shown in the following examples or by modifying the exemplified synthesis by means known to those of ordinary skill in the art.

2-[(Carboxymethyl)thio]ethyl N,N,N′,N′-tetrakis(2-chloroethyl)phosphorodiamidate. Thioglycolic acid (5.0 g, 54 mmol) was added slowly to an ice-cooled solution of crushed NaOH pellets (6.48 g, 162 mmol) in anhydrous methanol (20 mL), followed by anhydrous toluene (20 mL) and by 2-(4-bromobenzenesulfonyloxy)ethyl N,N,N′,N′-tetrakis(2-chloroethyl)phosphorodiamidate (66 mL of 0.82 M solution in toluene, 54 mmol), while maintaining the temperature of the reaction mixture below 5° C. After stirring for 30 minutes at 0-5° C., a white solid precipitated. The resulting white slurry was allowed to warm slowly to room temperature and stirred for an additional 18 hours. Analysis by LC/MS indicated that all the starting 2-(4-bromobenzenesulfonyloxy)ethyl N,N,N′,N′-tetrakis(2-chloroethyl)phosphorodiamidate had been consumed. The slurry was filtered and the filtrate was concentrated under vacuum to give a thick, off-white oil. This oil was partitioned between isopropyl acetate (100 mL) and 1N aqueous NaOH (100 mL). The isopropyl acetate solution was washed with 1N aqueous NaOH (2×100 mL). The combined aqueous solutions were extracted with isopropyl acetate (2×50 mL), acidified with 1N aqueous HCl to pH≅5, and extracted with isopropyl acetate (3×150 mL). The combined isopropyl acetate solutions were dried over anhydrous MgSO₄, filtered, and concentrated under vacuum to give a clear oil. Treatment of this oil with a small amount of ethyl ether caused precipitation of a white solid, which was filtered and dried under vacuum to give 2-[(carboxymethyl)thio]ethyl N,N,N′,N′-tetrakis(2-chloroethyl)-phosphorodiamidate as a white amorphous solid (18.5 g, 74% yield), 99% pure by HPLC-ELSD (Evaporative Light Scattering Detection). MS (ES⁻): m/z=461 [C₁₂H₂₃Cl₄N₂O₄PS—H];

¹H NMR (CDCl₃): δ=2.94-2.98 (m, 2H), 3.28 (s, 2H), 3.39-3.51 (m, 8H), 3.61-3.71 (m, 8H), 4.22-4.28 (m, 2H), carboxylate proton not seen; 31P NMR (CDCl₃): δ=17.53.

2-[(Carboxymethyl)sulfonyl]ethyl N,N,N′,N′-tetrakis(2-chloroethyl)phosphorodiamidate. A solution of 2-[(carboxymethyl)thio]ethyl N,N,N′,N′-tetrakis(2-chloroethyl)phosphorodiamidate (10.0 g, 21.22 mmol) in dichloromethane (20 mL) was cooled to 0° C. in an ice bath. Peracetic acid (32% by weight in acetic acid, 11.27 g, 47.4 mmol) was added slowly over 10 minutes. After the addition was complete, the reaction mixture was allowed to warm slowly to room temperature and stirring was continued for an additional 18 hours. Analysis by LC/MS indicated complete conversion to 2-[(carboxymethyl)sulfonyl]ethyl N,N,N′,N′-tetrakis(2-chloroethyl)phosphorodiamidate. The solvent was removed under vacuum and the oily residue was partitioned between ethyl acetate (300 mL) and saturated aqueous NaCl (100 mL). The ethyl acetate solution was decanted, washed with saturated aqueous NaHCO₃ (2×100 mL) and saturated aqueous NaCl (100 mL), and dried over anhydrous MgSO₄. The solids were filtered and the filtrate was evaporated under reduced pressure to give 2-[(carboxymethyl)sulfonyl]ethyl N,N,N′,N′-tetrakis(2-chloroethyl)phosphorodiamidate as an oil. Treatment of this oil with ethyl ether caused precipitation of a white solid, which was collected by filtration, washed with a small amount of ethyl ether, and dried under vacuum to give 2-[(carboxymethyl)sulfonyl]ethyl N,N,N′,N′-tetrakis(2-chloroethyl)phosphorodiamidate as a white amorphous solid (8.2 g, 77% yield), 99% pure by HPLC-ELSD.

MS (ES⁺): m/z=495 [C₁₂H₂₃Cl₄N₂O₆PS+H]; ¹H NMR (CDCl₃): δ=3.41-3.49 (m, 8H), 3.65-3.73 (m, 8H), 3.72 (t, 2H, J=4.5 Hz), 4.05 (s, 2H), 4.51-4.55 (m, 2H), carboxylated proton not seen; ³¹P NMR (CDCl₃): δ=17.64.

2-({2-Oxo-2-[(pyridin-3-ylmethyl)amino]ethyl}sulfonyl)ethyl N,N,N′,N′-tetrakis(2-chloroethyl)phosphorodiamidate hydrochloride. To a solution of 2-[(carboxymethyl)sulfonyl]ethyl N,N,N′,N′-tetrakis(2-chloroethyl)phosphorodiamidate (4.0 g, 8.06 mmol) in anhydrous dimethylformamide (10 mL) was added O-(benzotriazol-1-yl)-N,N,N′,N′-tetramethyluronium hexafluorophosphate (3.68 g, 9.7 mmol). The solution was allowed to stir at room temperature for 5 minutes and then treated with 3-(aminomethyl)pyridine (1.16 mL, 9.67 mmol) and N,N-diisopropylethylamine (1.70 mL, 8.06 mmol). The mixture was stirred at room temperature for 3 hours. Analysis by LC/MS and HPLC-ELSD showed that all the 2-[(carboxymethyl)sulfonyl]ethyl N,N,N′,N′-tetrakis(2-chloroethyl)phosphorodiamidate had been consumed. The mixture was diluted with ethyl acetate (75 mL) and washed with saturated aqueous NaHCO₃ (2×100 mL) and saturated aqueous NaCl (100 mL). The ethyl acetate layer was decanted, dried over anhydrous MgSO₄, and concentrated under reduced pressure to give 2-({2-oxo-2-[(pyridin-3-ylmethyl)amino]ethyl}sulfonyl)ethyl N,N,N′,N′-tetrakis(2-chloroethyl)-phosphorodiamidate as a white solid. The solid was dissolved in acetonitrile (20 mL), treated with 1M aqueous HCl (60 mL, 2 eq), and the resulting solution lyophilized to give 2-({2-oxo-2-[(pyridin-3-ylmethyl)amino]ethyl}sulfonyl)ethyl N,N,N′,N′-tetrakis(2-chloroethyl)-phosphorodiamidate hydrochloride as a colorless viscous oil (2.23 g, 75% yield), 99% pure by HPLC-ELSD. MS (ES⁺): m/z=585 [C₁₈H₂₉Cl₄N₄O₅PS+H];

¹H NMR (DMSO): δ=3.27-3.38 (m, 8H), 3.66-3.76 (m, 10H), 4.30-4.32 (m, 4H), 4.57 (d, 2H, J=6.0 Hz), 8.07-8.11 (m, 1H), 8.51 (d, 1H, J=8.0 Hz), 8.87 (d, 2H, J=6.0 Hz), 9.56-9.59 (m, 1H); ³¹P NMR (DMSO): δ=17.53.

2-({2-Oxo-2-[(pyridin-3-ylmethyl)amino]ethyl}sulfonyl)ethyl N,N,N′,N′-tetrakis(2-chloroethyl)phosphorodiamidate tartrate. 2-({2-Oxo-2-[(pyridin-3-ylmethyl)-amino]ethyl}sulfonyl)ethyl N,N,N′,N′-tetrakis(2-chloroethyl)phosphorodiamidate (6.0 g, 10.2 mmol) was dissolved in ethyl acetate (10 mL). This solution was added dropwise to a stirred 1.0M solution of tartaric acid in anhydrous ethanol (10.2 mL, 10.2 mmol). After the addition, the resulting mixture was stirred for 30 minutes, and then added dropwise to stirred diethyl ether (200 mL). The precipitate that formed was collected by filtration and dried under vacuum to give 2-({2-oxo-2-[(pyridin-3-ylmethyl)amino]ethyl}sulfonyl)ethyl N,N,N′,N′-tetrakis(2-chloroethyl)-phosphorodiamidate tartrate as an off-white solid (7.5 g, 99% yield), 98% pure by HPLC-ELSD.

The hydrochloride salt had a solubility in water of at least 10 mg/mL, and the tartrate salt had a solubility in water of at least 5 mg/mL.

The citrate, fumarate, methanesulfonate, and sulfate salts of 2-({2-oxo-2-[(pyridin-3-yl-methyl)amino]ethyl}sulfonyl)ethyl N,N,N′,N′-tetrakis(2-chloroethyl)phosphorodiamidate were also prepared as solids, and the maleate salt as an oil, by reacting 2-({2-oxo-2-[(pyridin-3-ylmethyl)-amino]ethyl}sulfonyl)ethyl N,N,N′,N′-tetrakis(2-chloroethyl)phosphorodiamidate with the corresponding acids using methods similar to those used to prepare the tartrate salt. Other salts of 2-({2-oxo-2-[(pyridin-3-ylmethyl)amino]ethyl}sulfonyl)ethyl N,N,N′,N′-tetrakis(2-chloroethyl)-phosphorodiamidate may similarly be prepared by using the appropriate acids, preferably in solvents that permit the isolation of the acid addition salts as solids.

### In Vitro Example 1

### Cytotoxicity/Growth Inhibition Assay

This example illustrates the beneficial effect of the compounds of this invention against human cancer cell lines in vitro. The results are considered predictive of efficacy in human cancer chemotherapy, as other anticancer agents tested in these assays have shown anticancer activity in humans.

The human cancer cell lines DLD-1 (colorectal adenocarcinoma), LNCaP (prostate carcinoma), and MIA PaCa-2 (pancreatic carcinoma) were obtained from the American Type Culture Collection, Manassas, Va., U.S.A., and MX-1 (breast carcinoma) from the National Cancer Institute, Bethesda, Md., U.S.A. The CellTiter-Glo assay kit was obtained from Promega Corporation, Madison, Wis., U.S.A. All products were used in accordance with manufacturer's directions. All assays were conducted in triplicate wells, with dimethyl sulfoxide (DMSO) solvent control. The extent of cell growth was expressed as a percentage of the signal from the solvent control wells.

Log-phase cells were trypsinized, collected by centrifugation, and resuspended in a small volume of fresh medium, and the density of viable cells was determined following Trypan Blue staining. Cells were diluted in fresh media (3×10³ cells/mL for DLD-1, MIA PaCa-2, and MX-1, and 6×10³ cells/mL for LNCaP), and added at 150 μL/well to 96-well plates, and incubated for several hours to allow attachment in the case of adherent cells. 2-({2-Oxo-2-[(pyridin-3-ylmethyl)-amino]ethyl}sulfonyl)ethyl N,N,N′,N′-tetrakis(2-chloroethyl)phosphorodiamidate hydrochloride, dissolved in DMSO, was diluted 50-fold with fresh medium and the diluted solutions immediately added at 50 μL/well to the cell suspensions, giving final compound concentrations between 0.1 μM and 200 μM and a final DMSO concentration of 0.5%. The cells were cultured for approximately three doubling times (3 days for MIA PaCa-2 and MX-1, and 4 days for DLD-1 and LNCaP). The cells were then collected by centrifugation, and 100 μL of the culture supernatant was replaced by the CellTiter-Glo reagent. After incubation for 10 minutes at room temperature, and the plate was read with a luminometer. 2-({2-Oxo-2-[(pyridin-3-ylmethyl)amino]ethyl}sulfonyl)ethyl N,N,N′,N′-tetrakis(2-chloroethyl)phosphorodiamidate hydrochloride showed the following IC₅₀ values in this assay: DLD-1, 12 μM; LNCaP, 67 μM; MIA PaCa-2, 16 μM; and MX-1, 22 μM. In each of these cell lines, 2-({2-oxo-2-[(pyridin-3-ylmethyl)amino]ethyl}sulfonyl)ethyl N,N,N′,N′-tetrakis(2-chloroethyl)phosphorodiamidate was at least as potent as canfosfamide.

### In Vivo Examples

In Vivo Example 1

### MX-1 Xenograft Assay, Intraperitoneal Administration

Female athymic nu/nu mice (Harlan, Indianapolis, Ind., U.S.A. or similar vendor), 6-8 weeks old (approximately 20 g), were implanted in the mammary fat pad of the right fore flank with 20-30 mg pieces of MX-1 tumor harvested from similar nu/nu mice that had previously been implanted with the MX-1 tumor. Approximately 7-10 days after tumor transplantation, when the tumor weight was approximately 50-200 mg, the mice were assigned to treatment groups such that each treatment group had a similar average tumor weight at the start of treatment. Groups of mice were treated with 2-({2-oxo-2-[(pyridin-3-ylmethyl)amino]ethyl}sulfonyl)ethyl N,N,N′,N′-tetrakis(2-chloroethyl)phosphorodiamidate hydrochloride at 50 and 100 mg/Kg, dissolved in aqueous 5% dextrose, by intraperitoneal injection once/day for 5 consecutive days, with vehicle control. Tumor mass, estimated from volume, was measured twice weekly; and tumor growth inhibition was measured when the average tumor mass in the control group first exceeded 2000 mg. 2-({2-Oxo-2-[(pyridin-3-ylmethyl)amino]ethyl}sulfonyl)ethyl N,N,N′,N′-tetrakis(2-chloroethyl)-phosphorodiamidate hydrochloride was active in this assay, causing 28% (at 50 mg/Kg) and 95% (at 100 mg/Kg) inhibition of tumor growth compared to vehicle.

### In Vivo Example 2

### MX-1 Xenograft Assay, Oral Administration

A study similar to that described in In vivo Example 1 was performed using oral administration of 2-({2-oxo-2-[(pyridin-3-ylmethyl)amino]ethyl}sulfonyl)ethyl N,N,N′,N′-tetrakis(2-chloroethyl)phosphorodiamidate hydrochloride. A group of mice was treated with the compound, dissolved in water, at 150 mg/Kg by gavage once/day for 5 consecutive days (with vehicle control). 2-({2-Oxo-2-[(pyridin-3-ylmethyl)amino]ethyl}sulfonyl)ethyl N,N,N′,N′-tetrakis(2-chloroethyl)phosphorodiamidate hydrochloride was active in this assay, causing 67% inhibition of tumor growth compared to vehicle.

### In Vivo Example 3

### MX-1 Xenograft Assay, Intravenous Administration

A study similar to that described in In vivo Example 1 was performed using intravenous administration of 2-({2-oxo-2-[(pyridin-3-ylmethyl)amino]ethyl}sulfonyl)ethyl N,N,N′,N′-tetrakis(2-chloroethyl)phosphorodiamidate hydrochloride. A group of mice was treated with the compound, dissolved in aqueous 5% dextrose, at 40 and 80 mg/Kg by tail vein injection once/day for 5 consecutive days (with vehicle control). 2-({2-Oxo-2-[(pyridin-3-ylmethyl)amino]-ethyl}sulfonyl)ethyl N,N,N′,N′-tetrakis(2-chloroethyl)phosphorodiamidate hydrochloride was active in this assay, causing 39% (at 40 mg/Kg) and 98% (at 80 mg/Kg) inhibition of tumor growth compared to vehicle.

### In Vivo Example 4

### MiaPaCa-2 Xenograft Assay, Intraperitoneal Administration

Male athymic nu/nu mice, 6-8 weeks old (approximately 20 g), were implanted subcutaneously in the right fore flank with 20-30 mg pieces of MIA PaCa-2 tumor harvested from similar nu/nu mice that had previously been implanted with the MIA PaCa-2 tumor. Approximately 7-10 days after tumor transplantation, when the tumor weight was approximately 50-200 mg, the mice were assigned to treatment groups such that each treatment group had a similar average tumor weight at the start of treatment. A group of mice was treated with 2-({2-oxo-2-[(pyridin-3-ylmethyl)-amino]ethyl}sulfonyl)ethyl N,N,N′,N′-tetrakis(2-chloroethyl)phosphorodiamidate hydrochloride, dissolved in aqueous 5% dextrose, at 100 mg/Kg by intraperitoneal injection once/day for 7 consecutive days, with vehicle control. 2-({2-Oxo-2-[(pyridin-3-ylmethyl)amino]ethyl}sulfonyl)-ethyl N,N,N′,N′-tetrakis(2-chloroethyl)phosphorodiamidate hydrochloride was active in this assay, causing 92% inhibition of tumor growth compared to vehicle.

2-({2-Oxo-2-[(pyridin-3-ylmethyl)amino]ethyl}sulfonyl)ethyl N,N,N′,N′-tetrakis(2-chloroethyl)phosphorodiamidate was safe and non-toxic at the doses tested.

### Formulation and Therapeutic Examples

### Formulation Example 1

### Formulation for Oral Administration

A solid formulation for oral administration is prepared by combining the following:

| | |
|---|---|
| Compound of this invention | 25.0% w/w |
| Magnesium stearate | 0.5% w/w |
| Starch | 2.0% w/w |
| Hydroxypropylmethylcellulose | 1.0% w/w |
| Microcrystalline cellulose | 71.5% w/w |

and the mixture is compressed to form tablets or filled into hard gelatin capsules containing, for example, 100 mg of the compound of this invention. Tablets may be coated, if desired, by applying a suspension of a film-forming agent (for example, hydroxypropylmethylcellulose), pigment (for example, titanium dioxide), and plasticizer (for example, diethyl phthalate), and drying the film by evaporation of the solvent.

### Formulation Example 2

### Formulation for IV Administration

A formulation for IV administration is prepared by dissolving a compound of this invention, for example as a pharmaceutically acceptable salt, to a concentration of 1% w/v in phosphate-buffered saline; and the solution is sterilized, for example by sterile filtration, and sealed in sterile containers containing, for example, 100 mg of a compound of this invention.

Alternatively, a lyophilized formulation is prepared by dissolving a compound of this invention, again for example as a pharmaceutically acceptable salt, in a suitable buffer, for example the phosphate buffer of the phosphate-buffered saline mentioned above, sterilizing the solution and dispensing it into suitable sterile vials, lyophilizing the solution to remove the water, and sealing the vials. The lyophilized formulation is reconstituted by the addition of sterile water, and the reconstituted solution may be further diluted for administration with a solution such as 0.9% sodium chloride intravenous infusion or 5% dextrose intravenous infusion.

### Therapeutic Example

### Therapy with Compounds of This Invention

A compound of this invention, diluted in 5% dextrose intravenous infusion, is administered intravenously over 30 minutes to a patient suffering from metastatic ovarian carcinoma at an initial dose of 100 mg/M²; and this dose is increased to 250 mg/M², 500 mg/M², 750 mg/M², and 1000 mg/m². The compound is administered at 1-week intervals. The same dose escalation is administered at 2- and 3-week intervals to other patients suffering from the same cancer.

While this invention has been described in conjunction with specific embodiments and examples, it will be apparent to a person of ordinary skill in the art, having regard to that skill and this disclosure, that equivalents of the specifically disclosed materials and methods will also be applicable to this invention; and such equivalents are intended to be included within the following claims.

We claim:

1. 2-({2-Oxo-2-[(pyridin-3-ylmethyl)amino]ethyl}sulfonyl)ethyl N,N,N′,N′-tetrakis(2-chloroethyl)phosphorodiamidate or its acid addition salt.

2. The compound of claim 1 in solid form.

3. The compound of claim 1 that is an acid addition salt of 2-({2-oxo-2-[(pyridin-3-ylmethyl)-amino]ethyl}sulfonyl)ethyl N,N,N′,N′-tetrakis(2-chloroethyl)phosphorodiamidate.

4. The compound of claim 3 in solid form.

5. The compound of claim 3 that is 2-({2-oxo-2-[(pyridin-3-ylmethyl)amino]ethyl}sulfonyl)-ethyl N,N,N′,N′-tetrakis(2-chloroethyl)phosphorodiamidate hydrochloride.

6. The compound of claim 3 that is 2-({2-oxo-2-[(pyridin-3-ylmethyl)amino]ethyl}sulfonyl)-ethyl N,N,N′,N′-tetrakis(2-chloroethyl)phosphorodiamidate tartrate.

7. The compound of claim 6 in solid form.

8. A pharmaceutical composition comprising a compound of claim 1 and an excipient.

9. A method of treating cancer in a human comprising administering to the human a compound of claim 1.

10. A method of preparing 2-({2-oxo-2-[(pyridin-3-ylmethyl)amino]ethyl}sulfonyl)ethyl N,N,N′,N′-tetrakis(2-chloroethyl)phosphorodiamidate or its acid addition salt, comprising:
(a1) reacting 2-[(carboxymethyl)sulfonyl]ethyl N,N,N′,N′-tetrakis(2-chloroethyl)-phosphorodiamidate with 3-(aminomethyl)pyridine to form 2-({2-oxo-2-[(pyridin-3-yl-methyl)amino]ethyl}sulfonyl)ethyl N,N,N′,N′-tetrakis(2-chloro ethyl)phosphorodiamidate, or
(a2) oxidizing 2-({2-oxo-2-[(pyridin-3-ylmethyl)amino]ethyl}thio)ethyl N,N,N′,N′-tetrakis(2-chloroethyl)phosphorodiamidate to form 2-({2-oxo-2-[(pyridin-3-yl-methyl)amino]ethyl}sulfonyl)ethyl N,N,N′,N′-tetrakis(2-chloroethyl)phosphorodiamidate;
optionally followed by one or more of:
(b) forming an acid addition salt of 2-({2-oxo-2-[(pyridin-3-ylmethyl)amino]ethyl}-sulfonyl)ethyl N,N,N′,N′-tetrakis(2-chloroethyl)phosphorodiamidate;
(c) converting an acid addition salt of 2-({2-oxo-2-[(pyridin-3-ylmethyl)amino]ethyl}-sulfonyl)ethyl N,N,N′,N′-tetrakis(2-chloroethyl)phosphorodiamidate to another acid addition salt of 2-({2-oxo-2-[(pyridin-3-ylmethyl)amino]ethyl}sulfonyl)ethyl N,N,N′,N′-tetrakis(2-chloroethyl)phosphorodiamidate; and
(d) converting an acid addition salt of 2-({2-oxo-2-[(pyridin-3-ylmethyl)amino]ethyl}-sulfonyl)ethyl N,N,N′,N′-tetrakis(2-chloroethyl)phosphorodiamidate to 2-({2-oxo-2-[(pyridin-3-ylmethyl)amino]ethyl}sulfonyl)ethyl N,N,N′,N′-tetrakis(2-chloroethyl)-phosphorodiamidate.

11. 2-({2-Oxo-2-[(pyridin-3-ylmethyl)amino]ethyl}thio)ethyl N,N,N′,N′-tetrakis(2-chloroethyl)phosphorodiamidate or its acid addition salt.

12. 2-[(Carboxymethyl)thio]ethyl N,N,N′,N′-tetrakis(2-chloroethyl)phosphorodiamidate.
